(19) United States
(12) Patent Application Publication
(10) Pub. No.: US 2025/0375482 A1
(43) Pub. Date: Dec. 11, 2025

(54) SLEEP-AIDING PROBIOTIC GOAT MILK POWDER AND PREPARATION METHOD THEREOF

(71) Applicant: SHAANXI UNIVERSITY OF SCIENCE AND TECHNOLOGY, Xi'an City (CN)

(72) Inventors: Guowei SHU, Xi'an City, (CN); Anxin MAO, Xi'an City, (CN); Guoliang LI, Xi'an, (CN); Qiqi ZHENG, Xi'an City, (CN); Qisheng HU, Xi'an, (CN); Xinru YU, Xi'an City (CN); Xu ZHANG, Xi'an (CN); Shiru DENG, Xi'an City (CN); Qian WANG, Xi'an City (CN); Qinfeng XU, Xi'an City (CN)

(21) Appl. No.: 19/223,700

(22) Filed: May 30, 2025

(30) Foreign Application Priority Data
Jun. 7, 2024 (CN) 202410739020.4

(51) Int. Cl. A61K 35/20; A61K 9/19; A61K 31/197; A61K 35/747

(52) U.S. Cl. CPC A61K 35/20; A61K 9/19; A61K 31/197; A61K 35/747

(57) ABSTRACT

A sleep-aiding probiotic goat milk powder and a preparation method thereof are provided. Goat milk is used as the raw material, and the goat milk is sterilized and cooled, then inoculated with Limosilactobacillus fermentum KD6, which produces the sleep-aiding functional factor γ-aminobutyric acid. The strain was deposited in the China Center for Type Culture Collection (CCTCC) on Sep. 7, 2023, with the deposit number CCTCC NO: M20231642. After being stirred evenly, the mixture is fermented at a constant temperature without the need to add precursors of γ-aminobutyric acid such as glutamic acid or sodium glutamate. Upon completion of fermentation, probiotic fermented goat milk containing γ-aminobutyric acid is obtained, and is then subjected to vacuum low-temperature spray drying to obtain the functional sleep-aiding probiotic goat milk powder.

### CROSS-REFERENCE TO RELATED APPLICATIONS

This application claims priority to Chinese Patent Application No. 202410739020.4, filed on Jun. 7, 2024, the contents of which are hereby incorporated by reference.

### INCORPORATION BY REFERENCE STATEMENT

This statement, made under Rules 77 (b) (5) (ii) and any other applicable rule incorporates into the present specification of an XML file for a “Sequence Listing XML” (see Rule 831 (a)), submitted via the USPTO patent electronic filing system or on one or more read-only optical discs (see Rule 1.52 (e) (8)), identifying the names of each file, the date of creation of each file, and the size of each file in bytes as follows:
File name: SequenceListing.xml
Creation date: May 28, 2025
Byte size: 3,200

### TECHNICAL FIELD

The present disclosure belongs to the technical field of fermentation engineering, and in particular to a sleep-aiding probiotic goat milk powder obtained by fermentation of goat milk with Limosilactobacillus fermentum KD6 and spray drying, and a preparation method thereof.

### BACKGROUND

Sleep, as a physiological activity crucial to human health, occupies approximately one-third of lifetime of a person. Sleep plays a key role in assisting the brain nervous system to store and restore energy, strengthening neuronal connections, and enhancing learning and memory. With the increasing number of insomnia patients and a trend toward younger demographics, consumer demand for sleep-aiding products has risen sharply. Currently, commercially available medicines are primarily represented by sedative-hypnotic medications such as benzodiazepines, which, while effective, may lead to side effects like medicine resistance and addiction with long-term use. Therefore, the search for and development of sleep-aiding foods to address insomnia through dietary therapy has become particularly urgent and necessary.

Melatonin is a commonly used sleep-aiding ingredient in health foods. It is an amine hormone produced by the pineal gland of mammals and humans, capable of illuminating melanin-producing cells, hence the name melatonin. Melatonin is a cosmetic hormone medicine that whitens the skin and delays aging. It may improve sleep, but there are issues such as low body temperature, excessive release of prolactin leading to infertility, and reduced male physiological desire.

γ-aminobutyric acid (GABA) is a natural active component and a non-protein functional amino acid. It is an important inhibitory neurotransmitter in the central nervous system of mammals, with biological functions such as calming nerves, regulating hormone secretion, improving lipid metabolism, and lowering blood pressure. Therefore, researching and developing foods rich in γ-aminobutyric acid is a necessary approach to addressing insomnia through dietary therapy.

### SUMMARY

To address the problems in the prior art, the present disclosure provides a sleep-aiding probiotic goat milk powder and a preparation method thereof. The method uses goat milk as the raw material, employs a food-grade strain/a strain that may be used for food-Limosilactobacillus fermentum KD6—for fermentation. This strain converts the glutamic acid naturally present in goat milk and the glutamic acid produced from protein decomposition into γ-aminobutyric acid. The fermented goat milk is then processed via low-temperature spray drying to obtain the sleep-aiding probiotic goat milk powder.

To achieve the above objective, the present disclosure provides the following solution: a Limosilactobacillus fermentum KD6, deposited at the China Center for Type Culture Collection (CCTCC), with the deposit number CCTCC NO: M20231642.

The disclosure further provides an application of Limosilactobacillus fermentum KD6 in treatment of insomnia, where Limosilactobacillus fermentum KD6 converts glutamic acid or sodium glutamate in a substrate containing glutamic acid or sodium glutamate into γ-aminobutyric acid.

In an embodiment, Limosilactobacillus fermentum KD6 converts glutamic acid or sodium glutamate in food containing glutamic acid or sodium glutamate into γ-aminobutyric acid.

In an embodiment, the food is goat milk.

The disclosure further provides a sleep-aiding probiotic fermented goat milk, where the freeze-dried bacterial powder of the Limosilactobacillus fermentum KD6 is added to the pre-treated goat milk, followed by constant-temperature fermentation at 34 degrees Celsius (° C.)−43° C. for 20 hours (h)-28 h to obtain the sleep-aiding probiotic fermented goat milk.

In an embodiment, the addition amount of the freeze-dried bacterial powder of Limosilactobacillus fermentum KD6 is 0.01%-0.05%, resulting in a γ-aminobutyric acid content of 0.488 grams per liter (g/L) to 0.633 g/L in the sleep-aiding probiotic fermented goat milk, a probiotic viable count of 2.32×10⁸ colony-forming units per milliliter (CFU/mL)-4.12×10⁸ CFU/mL, and a pH of 3.56-4.59.

The disclosure further provides a preparation method for the sleep-aiding probiotic fermented goat milk, including sterilizing goat milk at 90° C.-95° C. for 5-10 minutes (min), cooling to 36° C.-40° C., adding 0.01%-0.05% of the freeze-dried bacterial powder of Limosilactobacillus fermentum KD6, and fermenting at a constant temperature of 34° C.-43° C. for 20-28 h, and obtaining the sleep-aiding probiotic fermented goat milk.

The disclosure further provides a sleep-aiding probiotic fermented goat milk powder, where the sleep-aiding probiotic fermented goat milk or the sleep-aiding probiotic fermented goat milk prepared by the above method is subjected to vacuum low-temperature spray drying to obtain the sleep-aiding probiotic fermented goat milk powder.

In an embodiment, the sleep-aiding probiotic fermented goat milk powder has a γ-aminobutyric acid content of 3.561 grams per kilogram (g/kg)-4.648 g/kg and a probiotic viable count of 1.15×10⁸ colony-forming units per gram (CFU/g)-2.50×10⁸ CFU/g.

In an embodiment, the conditions of the vacuum low-temperature spray drying include an inlet air temperature of 65° C.-75° C., a feed peristaltic pump opening of 30%-50%, and a vacuum pressure of 0.03 megapascal (MPa)-0.04 MPa.

The disclosure discloses the following technical effects.

The disclosure provides a Limosilactobacillus fermentum KD6, deposited at the China Center for Type Culture Collection with the deposit number CCTCC NO: M20231642. The Limosilactobacillus fermentum KD6 may convert glutamic acid or sodium glutamate in substrates containing glutamic acid or sodium glutamate into γ-aminobutyric acid, thereby preparing sleep-aiding foods containing γ-aminobutyric acid.

The disclosure provides a sleep-aiding probiotic fermented goat milk. Unlike the method of directly adding γ-aminobutyric acid to conventional sleep-aiding foods, the disclosure introduces γ-aminobutyric acid into goat milk by fermenting goat milk with Limosilactobacillus fermentum KD6. The Limosilactobacillus fermentum KD6 converts free glutamic acid in goat milk and glutamic acid generated from protein hydrolysis into γ-aminobutyric acid, thereby enhancing the sleep-aiding functionality of the goat milk. Additionally, the fermentation by Limosilactobacillus fermentum KD6 produces metabolites such as lactic acid, which helps to reduce the gamy odor of the milk, improving its sensory evaluation.

In addition, the disclosure employs low-temperature spray drying technology to prepare the sleep-aiding probiotic fermented goat milk into the sleep-aiding probiotic fermented goat milk powder, and the low-temperature spray drying may maintain a high viable count in the goat milk powder. The goat milk powder produced contains up to 1.0×10⁸ CFU/g. Notably, no heat-resistant probiotic protectants or other additives are added during preparation, making the ingredient list simpler and more easily accepted by consumers. Overall, the sleep-aiding probiotic goat milk powder prepared by the disclosure is rich in γ-aminobutyric acid and probiotics, offering dual functions of aiding sleep and regulating intestinal flora balance.

### BRIEF DESCRIPTION OF THE DRAWINGS

FIG. 1 shows bacterial and colony morphology of Limosilactobacillus fermentum KD6.

FIG. 2 shows hemolysis of Limosilactobacillus fermentum KD6 and Escherichia coli.

FIG. 3 shows antibiotic sensitivity of Limosilactobacillus fermentum KD6.

FIG. 4 shows acid tolerance of Limosilactobacillus fermentum KD6.

FIG. 5 shows bile salt tolerance of Limosilactobacillus fermentum KD6.

FIG. 6 shows sodium chloride tolerance of Limosilactobacillus fermentum KD6.

FIG. 7 shows effect of Limosilactobacillus fermentum KD6 on γ-aminobutyric acid production in fermented goat milk.

FIG. 8 shows effects of inoculation amount, temperature, and duration on γ-aminobutyric acid production in goat milk fermented by Limosilactobacillus fermentum KD6.

### DETAILED DESCRIPTION OF THE EMBODIMENTS

To further explain the disclosure, the following describes the embodiments in conjunction with the drawings. These descriptions are intended to illustrate the features and advantages of the disclosure and are not intended to limit the scope of the claims.

### Embodiment 1: Isolation, Identification, and Probiotic Properties of Limosilactobacillus Fermentum KD6

### 1. Isolation of Limosilactobacillus Fermentum KD6

Milk kefir grains are inoculated at 3% into sterilized and cooled goat milk and fermented at room temperature for 24 hours (h), and then are used as samples. After a series of dilutions, and 30 strains of lactic acid bacteria are isolated using the plate spread method. Through repeated primary and secondary screening, a strain of lactobacillus with strong γ-aminobutyric acid production capacity is obtained.

### 2. Identification of Limosilactobacillus Fermentum KD6

16S ribosomal deoxyribonucleic acid (16S rDNA) is used for strain identification. After amplifying and purifying the target fragment, the strain is characterized based on the homology analysis of the partial 16S rDNA gene fragments. The measured sequence is compared and analyzed with the base sequence in the National Center for Biotechnology Information (NCBI) database, confirming that the strain is Limosilactobacillus fermentum. The base pair sequence is as follows:

CCGAGGAAGGGCGGACGGGTGAATTACCAGTAGGTAACCTGGCCCG
AAGCGGGGGGCTACCACCGGAAACCAATTGTAATTCCGCATAACAA
CGTTGTTCCCCAGAGTCCGAGTTAAAAAATGGGTTTTGGCTATCAC
TTTTGGATGGTCCCGCGGTGCATTAGCTAGTTGGTGGGGTAACGGC
TCACCAAGGCGATGATGCATAGCCGACTTGAGAGGGTAATCGGCCA
CAATGGGACTGAGACACGGCCCATACTCCTACGGGAGGCAGCAGTA
GGGAATCTTCCACAATGGGCGCAAGTCTGATGGAGCAACGCCGCGT
GAGTGAAGAAGGGTTTCGGCTCGTAAAACTCTGTTGTTAAAGAAGA
ACATATATGAGAGTAACTGTTCATACATTGACGGTATTTAACCAGA
AAGCCACGGCTAACTACGTGCCAGCAGCCGCGGTAATACGTAGGTG
GCAAGCGTTGTCCGGATTTATTGGGCGTAAAGCGAGTGCAGGCGGT
TTTTTAAGTCTGATGTGAAAGCCTTCGGCTCAACCGAAGAAGTGCA
TCGGAAACTGGAAAACTTGAGTGCAGAAGAGGGTAGTGGAACTCCA
TGTGTAGCGGTGGAAATGCGTAGATATATGGAAGAACACCAGTGGC
GAAGGCGGCTGTCTGGTCTGCAACTGACGCTGAGACTCGAAAGCAT
GGGTAGCGAACAGGATTAGATACCCTGGTAGTCCATGCCGTAAACG
ATGAATGCTAAGTGTTGGAGGGTTTCCGCCCTTCAGTGCTGCAGCT
AACGCATTAAGCATTCCGCCTGGGGAGTACGGCCGCAAGGTTGAAA
CTCAAAGGAATTGACGGGGGCCCGCACAAGCGGTGGAGCATGTGGT
TTAATTCGAAGCTACGCGAAGAACCTTACCAGGTCTTGACATCTTG
CGCCAATCTTAGAGATAGGGCGTTTCCTTCGGGAACGCAATGACAG
GTGGTGCATGGTTGTCGTCAGCTCGTGTCGTGAGATGTTGGGTTAA
GTCCCGCAACGAGCGCAACCCTTGTTATTAGTTGCCAGCATTAAGT
TGGGCACTCTGGTGAGACTGCCGGTGACAAACCGGAGGAAGGTGGG
GACGACGTCAGATCATCATGCCCCTTATGACCTGGGCTACACACGT
GCTACAATGGATGGTACAACGAGTTGCGAACTCGCGAGGGCAAGCA
AATCTCTTAAAACCGTTCTCAGTTCGGACTGCAGGCTGCAACTCGC
CTGCATGAAGTCGGAATCGCTAGTAATCGCGGATCAGCATGCCGCG
GTGAATACGTTCCCGGGCCTTGTACACACCGCCCAGTCACACCATG
AGAGTTTGTAACACCCAAAGTCGG

The strain is named Limosilactobacillus fermentum KD6 and deposited on Sep. 7, 2023 at the China Center for Type Culture Collection (CCTCC), Wuhan University, 299 Bayi Road, Wuchang District, Wuhan, Hubei Province. The deposit number is CCTCC NO: M20231642.

### Embodiment 2: Study on the Characteristics of Limosilactobacillus Fermentum KD6

### 1. Observation of Bacterial and Colony Morphology

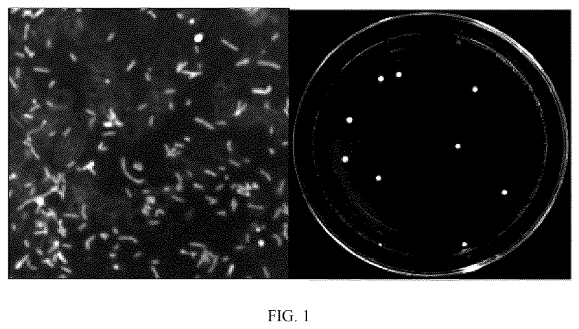

Limosilactobacillus fermentum KD6 is inoculated into de Man, Rogosa and Sharpe (MRS) broth at 1% inoculation amount and cultured at 37° C. for 24 h. Samples are taken for observation of bacterial and colony morphology. The bacterial cells are stained with toluidine blue for microscopic observation, and the colony morphology is observed using the dilution plate spread method. The bacterial and colony morphology are shown in FIG. 1.

As shown in FIG. 1, Limosilactobacillus fermentum KD6 is short rod-shaped, with some bacterial cells curved, no branches or spores, and the ends of the bacterial cells are mostly blunt circular. The colonies on MRS agar are convex, circular, with a milky white color, smooth and moist appearance, neat edges, and easily picked, all of which conform to the morphological characteristics of lactic acid bacteria colonies.

### 2. Comparison of Probiotic Properties

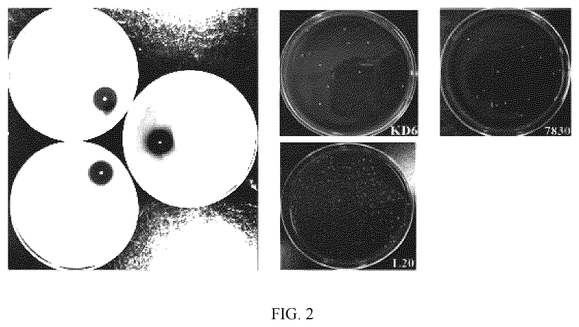

The hemolysis, antibiotic sensitivity, and tolerance to acid, bile salts, and sodium chloride of three probiotic strains, namely Limosilactobacillus fermentum KD6, the publicly known Lactiplantibacillus plantarum 7830 and Lacticaseibacillus rhamnosus L20 are compared. The results are shown in FIG. 2-FIG. 6.

As shown in FIG. 2, clear transparent halos, namely β-hemolysis are observed on Columbia blood agar plates inoculated with hemolytic Escherichia coli, while no hemolysis is observed on Columbia blood agar plates inoculated with the three probiotic strains, indicating that all three strains are non-hemolytic and are safe strains. The lack of hemolysis ensures the safety of the strains and is used as one of the screening criteria for probiotic strains.

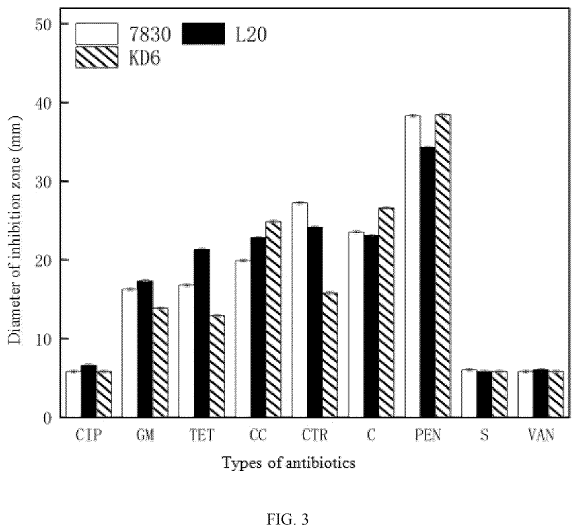

As shown in FIG. 3, the three probiotic strains are generally sensitive to penicillin, with inhibition zones ranging from 31.26 millimeters (mm) to 44.38 mm, and resistant to ciprofloxacin, streptomycin, and vancomycin. However, significant differences in resistance are observed among different strains. For ciprofloxacin (CIP), KD5 shows significantly higher sensitivity than the other strains (p<0.05), with an inhibition zone of 10.71 mm, while other strains are generally resistant. For gentamicin (GM), KD11 is sensitive (p<0.05), with an inhibition zone of 24.72 mm. For tetracycline (TET), KD5 is sensitive (p<0.05), with an inhibition zone of 28.71 mm. For clindamycin (CC), KD5 is sensitive (p<0.05), with an inhibition zone of 27.31 mm. For ceftriaxone (CTR), KD11 is sensitive (p<0.05) and significantly higher than the other strains, with an inhibition zone of 44.38 mm. For chloramphenicol (C), KD11 is sensitive (p<0.05), with an inhibition zone of 36.38 mm.

Zhang Zhuoyang et al. studied the antibiotic sensitivity of multiple lactic acid bacteria strains and found that most tested lactic acid bacteria have inherent resistance to streptomycin and vancomycin, and this resistance is generally non-transferable to pathogenic bacteria. Guo et al. studied the resistance of Lactobacillus delbrueckii subsp. Bulgaricus isolated from traditional dairy products, and the results showed that all Lactobacillus delbrueckii subsp. Bulgaricus were sensitive to gentamicin, clindamycin, tetracycline, and chloramphenicol, but exhibited different resistance to ciprofloxacin, streptomycin, and vancomycin, which is consistent with the conclusion of the disclosure.

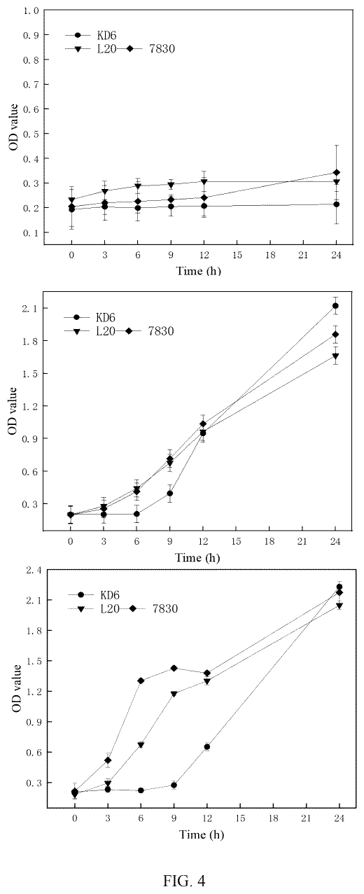

Acid tolerance is an important criterion for selecting probiotic strains. The pH value of human gastric juice is constantly changing, typically around 3.0, and may reach 1.8 under fasting conditions. Probiotics must survive the extremely acidic environment of gastric juice to exert the positive probiotic effects in the human body. The acid tolerance results of the three strains are shown in FIG. 4.

In FIG. 4, the growth of probiotics is inhibited in acidic environments, with the inhibition becoming more pronounced as the pH decreased. When the pH drops to 5, all three strains of probiotics show strong tolerance, generally following the “S”-shaped microbial growth pattern. After 24 h of fermentation, the final OD_{600 nm} values exceed 2.0. When the pH drops to 4, there is little difference in the tolerance of the three probiotics, with OD_{600 nm} values ranging from 1.663 to 2.122. The acid resistance ranking is: KD6>7830>L20, where the growth curve of KD6 has a relatively small impact and exhibits strong acid resistance. When the pH drops to 3, all three strains grow slowly. Chen Junfei et al. isolated 6 strains of lactic acid bacteria from pickles for acid resistance testing. The results showed that all strains had the highest OD_{600 nm} value at pH 5.5, and almost no strain growth at pH≤4. In comparison, the three strains in the present disclosure have overall stronger acid resistance, which may be due to the poorer acid resistance of the heterologous fermentation lactic acid bacteria isolated from pickles compared to the homologous fermentation lactic acid bacteria. Studies indicate that strains from different sources have varying degrees of tolerance to the human digestive tract, and the effects of probiotics are strain specific. Aiba et al. reported a strain of Lactobacillus johnsonii No. 1088 isolated from human gastric juice, which may maintain a 10% survival rate even after being treated for 2 hours in a pH 1.0 environment.

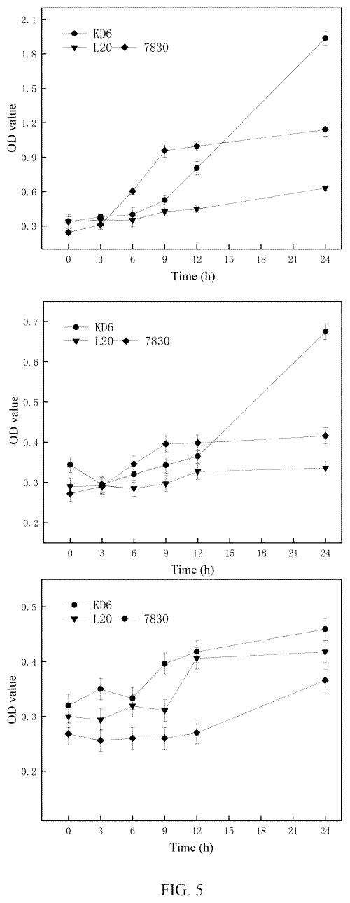

After being processed by gastric juice, food enters the duodenum and comes into full contact with bile salts that have antibacterial properties. Bile salts may cause strain death by disrupting the integrity of cell membranes. Therefore, the tolerance of probiotics to high concentrations of bile salts in the human gut is an important indicator for screening probiotics. The tolerance results of all three strains to bile salts are shown in FIG. 5.

As shown in FIG. 5, with the increase of bile salt concentration, the growth of bacterial cells is more affected. At a bile salt concentration of 0.1%, the OD 600 nm values range from 0.271 to 1.936, with KD6 exhibiting strong bile salt tolerance and a final OD_{600 nm} value of 1.936 after 24 h of culture. Strain 7830 shows some tolerance in the early stage (before 12 h), but its growth trend gradually slows down with prolonged treatment duration. At a bile salt concentration of 0.2%, the OD_{600 nm} values range from 0.221 to 0.661, with the tolerance ranking being KD6>7830>L20, indicating that KD6 maintains good tolerance even at higher bile salt concentrations. Studies show that 0.3% bile salt is the maximum concentration in the intestines of healthy adult males, and food typically takes 1˜4 h to pass through. After 12 h of treatment at this concentration, all three strains of probiotics exhibit a certain tolerance, showing a slow overall growth trend, with KD6 achieving a final OD_{600 nm} value of 0.459 after 24 h of culture. Reuben et al. point out that the bile salt tolerance concentration of lactic acid bacteria with probiotic potential needs to reach 0.3%. All three strains in the present disclosure meet the requirements.

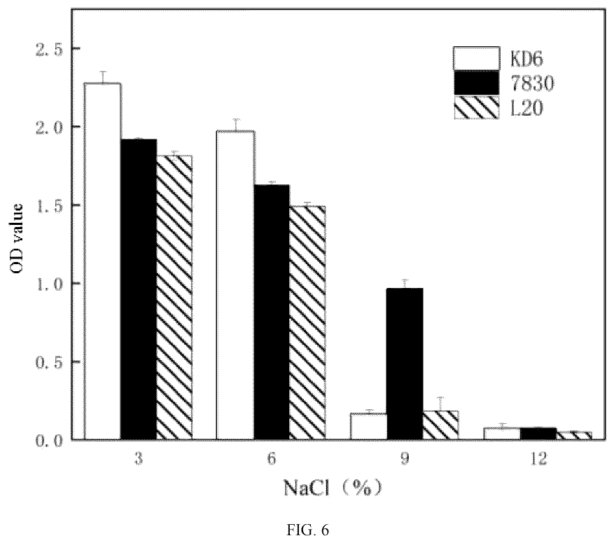

Probiotics need to pass through the stomach and small intestine before finally colonizing in the large intestine in order to exert their probiotic effects. Various osmotic pressures are also key factors that bacteria need to tolerate. The effect of adding different concentrations of sodium chloride to MRS broth on the growth of three strains is shown in FIG. 6.

Wu Yating et al. screened three Lactiplantibacillus plantarum strains with strong osmotic pressure tolerance from naturally fermented chili paste, capable of tolerating up to 7% NaCl concentration. In comparison, strain 7830 in the present disclosure exhibits even stronger osmotic pressure tolerance. However, some studies suggest that the osmotic pressure tolerance of probiotics is influenced by their long-term environment, so probiotics may adapt to stable changes in osmotic pressure. However, sudden increases or decreases in osmotic pressure may greatly inhibit the growth of probiotics and even lead to death.

As shown in FIG. 6, the OD_{600 nm} values of the strains decrease with the increase of NaCl content. When the mass fraction of NaCl is 3%, the growth of lactic acid bacteria is basically not affected, with OD_{600 nm} values ranging from 1.727 to 2.272, and KD6 showing significantly higher acid tolerance than the other strains (p<0.05). When the mass fraction of NaCl is 6%, the growth of other strains is significantly inhibited, with OD_{600 nm} values ranging from 1.49 to 1.622, but KD6 has a higher OD_{600 nm} value of 1.968 (p<0.05). When the mass fraction of NaCl is greater than 6%, the growth of lactic acid bacteria is extremely slow, with only 7830 showing a certain growth trend (p<0.05), and the OD_{600 nm} values are 0.965. When the mass fraction of NaCl is 12%, the OD_{600 nm} values of the strains are all below 0.100.

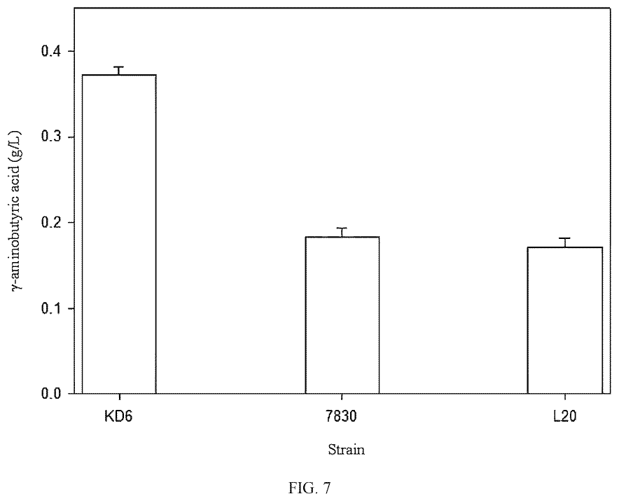

The third-generation activated strains are inoculated into goat milk at a 5% inoculation amount. After 24 h of constant temperature fermentation at 37° C., the whey is separated and the GABA content is determined using the Berthlot colorimetric method, and the γ-aminobutyric acid concentration is found to be in the range of 0-1.0 microgram per milliliter (μg/mL). The standard curve equation is y=2.0686x+0.0804, and the regression equation coefficient R² is 0.9968, indicating a strong linear relationship between absorbance and γ-aminobutyric acid concentration. This linear relationship range may be used to determine γ-aminobutyric acid concentration. The GABA production results for the three strains are shown in FIG. 7. It may be seen from FIG. 7 that without adding sodium glutamate to the goat milk, all three strains of probiotics convert high concentrations of GABA, attributed to the high activity of glutamic acid decarboxylase in probiotics, which converts free glutamic acid in goat milk or glutamic acid produced by protein hydrolysis into GABA. After 24 h of fermentation at 37° C., the range of GABA production in goat milk is 0.1709 to 0.3718 g/L, with KD6 producing significantly higher GABA than the other strains (p<0.05). Sun Shixin et al. reported Lactococcus lactis subsp. lactis 4043, which was inoculated at a 3% inoculation amount in a milk-based substrate medium containing 2 g/L sodium glutamate. After 48 h of fermentation at 30° C., the strain produced approximately 0.39 g/L of GABA. Nejati F. et al. cultured Lactococcus lactis DBCA and Lactiplantibacillus plantarum PU11 in sterilized condensed milk, achieving a GABA concentration of 0.14 g/L after 48 h of co-fermentation at 37° C. By comparison, it may be seen that the GABA ability prepared by KD6 transformation belongs to a relatively high level.

### Embodiment 3: Preparation of Sleep-Aiding Probiotic Fermented Goat Milk and Goat Milk Powder

### 1. Effects of Fermentation Conditions on GABA Production in Goat Milk Fermented by Limosilactobacillus Fermentum KD6

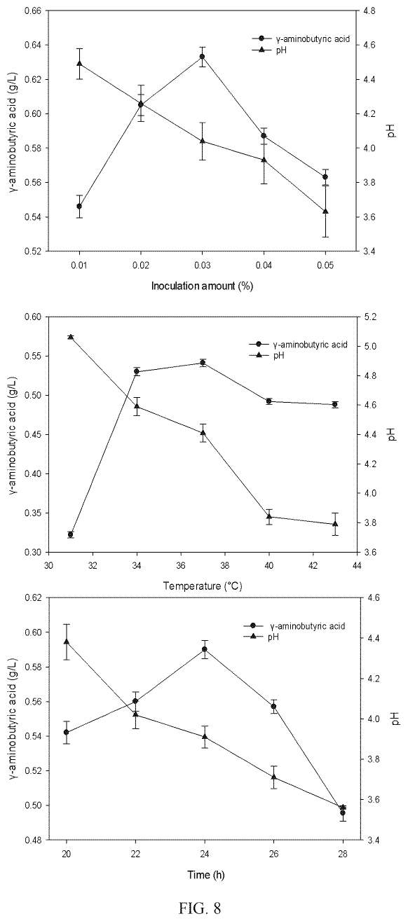

The freeze-dried bacterial powder of Limosilactobacillus fermentum KD6 is inoculated into goat milk as a starter to study the effects of inoculation amount (0.01%, 0.02%, 0.03%, 0.04%, 0.05%, weight per volume (w/v)), fermentation temperature (31° C., 34° C., 37° C., 40° C., 43° C.), and fermentation duration (20 h, 22 h, 24 h, 26 h, 28 h) on GABA production in goat milk fermented by Limosilactobacillus fermentum KD6. The pH and γ-aminobutyric acid content of the fermented goat milk are measured. The basic experimental conditions are fermentation duration of 20 h, temperature of 37° C., and inoculation amount of 0.01%. As shown in FIG. 8, with the increase of inoculation amount, the γ-aminobutyric acid content in goat milk fermented by Limosilactobacillus fermentum KD6 first increases and then decreases, and the pH gradually decreases, with γ-aminobutyric acid content ranging from 0.546 g/L to 0.633 g/L. As the fermentation temperature increases, the γ-aminobutyric acid content in goat milk fermented by Limosilactobacillus fermentum KD6 first increases and then decreases to tend to remain unchanged, and the pH rapidly decreases and then tends to remain unchanged, with γ-aminobutyric acid content ranging from 0.488 g/L to 0.541 g/L. As the fermentation duration prolongs, the γ-aminobutyric acid content in goat milk fermented by Limosilactobacillus fermentum KD6 first increases and then decreases to tend to remain unchanged, and the pH gradually decreases, with γ-aminobutyric acid content ranging from 0.494 g/L to 0.590 g/L. Targeting a γ-aminobutyric acid content of not less than 0.45 g/L, the suitable conditions for fermenting goat milk with Limosilactobacillus fermentum to produce γ-aminobutyric acid are: inoculation amount of 0.01% to 0.05%, temperature of 34° C.-43° C., and fermentation duration of 20 to 28 h.

According to the results of fermentation conditions, two batches of samples of sleep-aiding probiotic fermented goat milk, A and B, are obtained under fermentation condition A with an inoculation amount of 0.03% and culture at 37° C. for 24 h, and fermentation condition B with an inoculation amount of 0.01% and culture at 43° C. for 20 h. After sampling and determination, the viable counts are 4.12×10⁸ CFU/mL and 2.32×10⁸ CFU/mL respectively, and the GABA levels are 0.626 g/L and 0.504 g/L respectively. Then, the samples are dried by vacuum low-temperature spray (vacuum low-temperature spray dryer, BILON-VSD1500, Bilon Company) to prepare the sleep-aiding probiotic goat milk powder, with high-temperature spray drying (small spray dryer, YM-6000Y, Shanghai Yuming Instrument Co., Ltd.) as a control. The high-temperature spray drying conditions are an inlet air temperature of 170° C., outlet temperature of 85° C., while the vacuum low-temperature spray drying conditions are an inlet air temperature of 65° C. and an outlet temperature of 75° C., peristaltic pump openings of 30%-50%, and drying vacuum pressures of 0.03-0.04 MPa. The results are shown in Table 1:

TABLE 1
Effects of high-temperature and vacuum low-temperature spray drying
on probiotic viable count and activity of antihypertensive peptide

| | | | Vacuum low-temperature spray | | |
|---|---|---|---|---|---|
| | | | drying | | |
| | | | 65° C. | 65° C. | 75° C. |
| | | High-temperature spray | (Pump | (Pump | (Pump |
| Indicator | | drying | 30%) | 40%) | 50%) |
| Sample | Viable count/ | 0.023 | 2.50 | 2.39 | 2.16 |
| A | 10⁸ CFU/g | | | | |
| | Survival rate/% | 0.56 | 61.09 | 58.23 | 52.72 |
| | GABA | 3.496 | 4.648 | 4.552 | 4.472 |
| | content/g/kg | | | | |
| Sample | Viable count/ | 0.011 | 1.39 | 1.23 | 1.15 |
| B | 10⁸ CFU/g | | | | |
| | Survival rate/% | 0.48 | 59.85 | 53.22 | 49.62 |
| | GABA | 2.873 | 3.758 | 3.634 | 3.561 |
| | content/g/kg | | | | |

As shown in Table 1, the sleep-aiding probiotic fermented goat milk samples A and B, after vacuum low-temperature spray drying, yield sleep-aiding probiotic goat milk powder with viable counts exceeding 1.0×10⁸ CFU/g, ranging from 1.15×10⁸ CFU/g to 2.50×10⁸ CFU/g, survival rates of probiotics higher than 45%, ranging from 49.2% to 61.09%, and GABA contents higher than 3.5 g/kg, ranging from 3.561 g/kg to 4.648 g/kg. In contrast, the high-temperature spray drying group has viable counts ranging from 1.1×10⁶ CFU/g to 2.3×10⁶ CFU/g, survival rates below 1%, and reduced GABA content. Thus, vacuum low-temperature spray drying is suitable for preparing sleep-aiding probiotic goat milk powder.

1. A sleep-aiding probiotic fermented goat milk, wherein freeze-dried bacterial powder of Limosilactobacillus fermentum KD6 strain with an addition amount of 0.01%-0.05% is added to pre-treated goat milk, followed by constant-temperature fermentation at 34° C.-43° C. for 20 h-28 h to obtain the sleep-aiding probiotic fermented goat milk;
wherein the Limosilactobacillus fermentum KD6 strain is deposited at the China Center for Type Culture Collection under deposit number CCTCC NO: M20231642.

2. The sleep-aiding probiotic fermented goat milk according to claim 1, wherein a γ-aminobutyric acid content in the sleep-aiding probiotic fermented goat milk is 0.488 g/L to 0.633 g/L, a probiotic viable count is 2.32×10⁸ CFU/mL-4.12×10⁸ CFU/mL, and a pH is 3.56-4.59.

3. A preparation method for a sleep-aiding probiotic fermented goat milk, wherein goat milk is sterilized at 90° C.-95° C. for 5 min-10 min, cooled to 36° C.-40° C., and then added with 0.01%-0.05% of freeze-dried bacterial powder of Limosilactobacillus fermentum KD6 strain, followed by constant-temperature fermentation at 34° C.-43° C. for 20 h-28 h to obtain the sleep-aiding probiotic fermented goat milk;
wherein the Limosilactobacillus fermentum KD6 strain is deposited at the China Center for Type Culture Collection under deposit number CCTCC NO: M20231642.

4. A sleep-aiding probiotic fermented goat milk powder, comprising: goat milk powder and freeze-dried bacterial powder of Limosilactobacillus fermentum KD6 strain, wherein the Limosilactobacillus fermentum KD6 strain is deposited at the China Center for Type Culture Collection under deposit number CCTCC NO: M20231642.

5. The sleep-aiding probiotic fermented goat milk powder according to claim 4, wherein the sleep-aiding probiotic fermented goat milk powder has a γ-aminobutyric acid content of 3.561 g/kg-4.648 g/kg and a probiotic viable count of 1.15×10⁸ CFU/g-2.50×10⁸ CFU/g.


SEQUENCE LISTING (1 sequence)

SEQ ID NO: 1 - DNA, 1358 bp, synthetic construct
  source 1..1358: mol_type = other DNA
ccgaggaagggcggacgggtgaattaccagtaggtaacctggcccgaagcggggggctaccaccggaaaccaattgtaattccgcataacaacgttgttccccagagtccgagttaaaaaatgggttttggctatcacttttggatggtcccgcggtgcattagctagttggtggggtaacggctcaccaaggcgatgatgcatagccgacttgagagggtaatcggccacaatgggactgagacacggcccatactcctacgggaggcagcagtagggaatcttccacaatgggcgcaagtctgatggagcaacgccgcgtgagtgaagaagggtttcggctcgtaaaactctgttgttaaagaagaacatatatgagagtaactgttcatacattgacggtatttaaccagaaagccacggctaactacgtgccagcagccgcggtaatacgtaggtggcaagcgttgtccggatttattgggcgtaaagcgagtgcaggcggttttttaagtctgatgtgaaagccttcggctcaaccgaagaagtgcatcggaaactggaaaacttgagtgcagaagagggtagtggaactccatgtgtagcggtggaaatgcgtagatatatggaagaacaccagtggcgaaggcggctgtctggtctgcaactgacgctgagactcgaaagcatgggtagcgaacaggattagataccctggtagtccatgccgtaaacgatgaatgctaagtgttggagggtttccgcccttcagtgctgcagctaacgcattaagcattccgcctggggagtacggccgcaaggttgaaactcaaaggaattgacgggggcccgcacaagcggtggagcatgtggtttaattcgaagctacgcgaagaaccttaccaggtcttgacatcttgcgccaatcttagagatagggcgtttccttcgggaacgcaatgacaggtggtgcatggttgtcgtcagctcgtgtcgtgagatgttgggttaagtcccgcaacgagcgcaacccttgttattagttgccagcattaagttgggcactctggtgagactgccggtgacaaaccggaggaaggtggggacgacgtcagatcatcatgccccttatgacctgggctacacacgtgctacaatggatggtacaacgagttgcgaactcgcgagggcaagcaaatctcttaaaaccgttctcagttcggactgcaggctgcaactcgcctgcatgaagtcggaatcgctagtaatcgcggatcagcatgccgcggtgaatacgttcccgggccttgtacacaccgcccagtcacaccatgagagtttgtaacacccaaagtcgg